(19) United States
(12) Patent Application Publication
(10) Pub. No.: US 2019/0134430 A1
(43) Pub. Date: May 9, 2019

(54) ULTRASONIC CARTRIDGE AND HEAD FOR ULTRASOUND THERAPY USING SAME

(71) Applicant: CLASSYS INC., Seoul (KR)

(72) Inventor: Seong Jai Jeong, Seoul (KR)

(73) Assignee: CLASSYS INC., Seoul (KR)

(21) Appl. No.: 16/302,155

(22) PCT Filed: May 15, 2017
(86) PCT No.: PCT/KR2017/005013

§ 371 Date: Nov. 16, 2018

(30) Foreign Application Priority Data
May 16, 2016 (KR) 10-2016-0059514

(51) Int. Cl. A61N 7/02; B06B 1/00

(52) U.S. Cl. CPC A61N 7/02; A61N 2007/0008; B06B 1/00

(57) ABSTRACT

The present invention relates to an ultrasonic cartridge and a head for ultrasound therapy using the same. A cartridge housing, which has an ultrasonic transducer unit therein, stores a liquid, and has an ultrasonic wave transmission window on the lower surface thereof, is divided into an upper housing member provided with the ultrasonic transducer unit and a lower housing member provided with the ultrasonic wave transmission window, and a distance between the ultrasonic transducer unit and the ultrasonic wave transmission window is adjusted, such that the depth of focus of ultrasonic waves in the skin can be controlled. As such, the structure of a product can be significantly simplified, and the manufacturing cost can be considerably reduced.

### TECHNICAL FIELD

The present invention relates generally to an ultrasonic cartridge and a head for ultrasound therapy using the same. More particularly, the present invention relates to an ultrasonic cartridge and a head for ultrasound therapy using the same, which are applied to an obesity treatment apparatus using high intensity focused ultrasound (HIFU) and are capable of adjusting a depth of focus of the ultrasound.

### BACKGROUND ART

In recent years, with the westernization of dietary habits, obesity has rapidly increased, leading to a major cause of harming public health and beauty. Thus, various diet programs and obesity treatment ultrasonic apparatuses have been developed and widely used to treat obesity

An obesity treatment technology using a high intensity focused ultrasound (HIFU) technology has been used primarily for anticancer treatment, in which cancer cells are destroyed by selectively coagulating tumors in internal organs at high temperature in a non-invasive way, but Solta Medical Inc. in U.S. has firstly developed the equipment for treatment of abdominal obesity in the human body called Liposonix equipped with HIFU. The process of fat destruction using high intensity focused ultrasound (HIFU) is performed in such a way that when the focused ultrasound is focused at a certain point on the adipocyte, the tissue temperature instantaneously rises from 65° C. to 100° C., thereby destroying the tissue. Unlike other dermatological equipment such as laser and RF high frequency equipment, the HIFU equipment concentrates HIFU energy on the non-invasively selected area without causing any damage to the skin surface, thereby inducing fat to cause coagulation necrosis.

As an obesity treatment ultrasonic apparatus, there is an “Apparatus and method for destruction of adipose tissue” disclosed in Korean Patent Application Publication No. 10-2007-7027898. The apparatus disclosed in Korean Patent Application Publication No. 10-2007-7027898 includes: a head having at least one ultrasonic transducer; a suspension for supporting the head; and a controller for controlling the ultrasonic transducer, thereby treating obesity in a non-invasive way by deforming the tissue of subcutaneous fat with ultrasonic energy. Further, a “Tissue imaging and treatment system”, which has been disclosed in Korean Patent Application Publication No. 10-2011-7017983, includes: a hand wand that is manually operated; and a detachable emitter-receiver module having an ultrasonic transducer. The system is configured to display a skin image in an imaging mode through a display connected to the hand wand, and make skin elastic and remove wrinkles by radiating ultrasonic waves to the portion of 3 mm to 4.5 mm (SMAS layer) under the skin in a treatment mode.

Meanwhile, those obesity treatment ultrasonic apparatuses are provided with a head or a handpiece that generates HIFU according to control of a controller to decompose the subcutaneous fat of a patient.

In a head for ultrasound therapy for obesity treatment, to improve efficiency of ultrasound therapy and to facilitate treatment, the techniques of moving an ultrasonic transducer have been proposed, and prior arts for moving an ultrasonic transducer include the following patents.

An “Ultrasound therapy head with movement control” disclosed in Korean Patent Application Publication No. 10-2006-7013272 includes: an enclosure having a window; an HIFU transducer suspended in the enclosure; and an actuator configured to move the HIFU transducer in X and Y-axial direction such that the HIFU transducer provides energy to subcutaneous fat through the window using HIFU. Further, a “Therapy head using ultrasound system” disclosed in Korean Patent Application Publication No. 10-2010-7019552 includes: an enclosure; a partition separating a lower compartment provided with an window, and an upper compartment; an opening of the partition; a control arm provided in the opening and configured to be movable; an operation assembly disposed in the upper compartment and configured to control the control arm; and an HIFU transducer coupled to the lower end of the control arm.

Further, a “Three-axis control head of Non-invasive subcutaneous fat reduction apparatus using HIFU” disclosed in Korean Patent No. 10-1177691 by the applicant of the present invention is configured such that the movement of the ultrasonic transducer is controlled in three axes by control of a controller, thereby providing ultrasonic waves three-dimensionally to a fat layer of a subject.

Further, a “Cartridge with adjustable depth for ultrasonic handpiece and ultrasonic handpiece including the same” disclosed in Korean Patent No. 1578490 is configured such that ultrasonic head for ultrasonic obesity therapy linearly reciprocates an ultrasonic transducer, thereby adjusting the depth of focus of the ultrasound.

However, the conventional head for ultrasound therapy is problematic in that since an actuator for directly moving the ultrasonic transducer is required to adjust the depth of focus of the ultrasound in the skin, the structure is complicated and the manufacturing cost is high.

### DISCLOSURE

### Technical Problem

Accordingly, the present invention has been made keeping in mind the above problems occurring in the prior art, and an object of the present invention is to provide an ultrasonic cartridge and a head for ultrasound therapy using the same, in which in the state where the position of an ultrasonic transducer is fixed in the upward and downward direction, a distance between the contact surface with skin and the ultrasonic transducer is adjusted, thereby simply adjusting the depth of focus of the ultrasound.

### Technical Solution

In order to accomplish the above object, the present invention provides an ultrasonic cartridge including: a cartridge housing provided at a lower portion thereof with an ultrasonic wave transmission window through which ultrasonic waves are transmitted, and configured such that a liquid is stored therein; and an ultrasonic transducer unit provided in the cartridge housing, and configured to provide ultrasonic waves to a treatment target area through the ultrasonic wave transmission window and form a focus on the treatment target area, wherein the cartridge housing includes an upper housing member in which the ultrasonic transducer unit is provided, and a lower housing member in which the ultrasonic wave transmission window is provided, and is configured to adjust a distance between the ultrasonic transducer unit and the ultrasonic wave transmission window.

In the present invention, the lower housing member may be moveably coupled to the upper housing member to adjust the distance between the ultrasonic transducer unit and the ultrasonic wave transmission window.

In the present invention, the lower housing member may be moveable in a vertical direction based on a reference plane when a contact surface of a lower surface of the cartridge housing brought in contact with skin to be treated or a skin surface of the treatment target area is set as the reference plane.

In the present invention, the lower housing member may be threaded with the upper housing member to be moved according to a rotational direction.

In the present invention, the upper housing member may include an upper surface portion in which the ultrasonic transducer unit is provided, and a first side portion protruding downward from an outer edge of the upper surface portion, the lower housing member may include a lower surface portion in which the ultrasonic wave transmission window is provided, and a second side portion protruding upward from an outer edge of the lower surface portion to be overlapped with at least a part of the first side portion, and a sealing O-ring member may be inserted between the first side portion and the second side portion.

In the present invention, a stop groove may be provided in one of the first side portion and the second side portion, a stopping portion may be provided in a remaining one of the first side portion and the second side portion to be stopped by being engaged with the stop groove, and at least one of the stop groove and the stopping portion may be provided in plural to be spaced apart from each other in a moving direction of the lower housing member.

In the present invention, a stopper protrusion may be provided in one of the first side portion and the second side portion to be stopped at an uppermost end and a lowermost end of the lower housing member, and a stopper stopping portion may be provided in a remaining one of the first side portion and the second side portion to be stopped by the stopper protrusion at the uppermost end and the lowermost end of the lower housing member.

In the present invention, the upper housing member may be provided with a pressure regulating valve to regulate internal pressure when the lower housing member is moved.

In the present invention, the pressure regulating valve may be configured to introduce air into the upper housing member when the lower housing member is moved in a direction of increasing the distance between the ultrasonic transducer unit and the ultrasonic wave transmission window, and discharge air in the upper housing member to an outside when the lower housing member is moved in a direction of decreasing the distance between the ultrasonic transducer unit and the ultrasonic wave transmission window.

In order to accomplish the above object, the present invention provides a head for ultrasound therapy, the head including: an ultrasonic cartridge according to the present invention; and a head body configured such that the ultrasonic cartridge is detachably coupled to a lower portion thereof, and formed in a shape that allows an operator to grip the head body.

### Advantageous Effects

According to the present invention configured as described above, a distance between the contact surface with skin and the ultrasonic transducer is adjusted with a simple housing structure, without an actuator, thereby adjusting the depth of focus of the ultrasound in the skin, and thus, it is possible to significantly simplify the structure of the product and possible to considerably reduce the manufacturing cost.

### DESCRIPTION OF DRAWINGS

FIG. 1 is a sectional view showing an embodiment of an ultrasonic cartridge according to the present invention;

FIG. 2 is a sectional view showing another embodiment of an ultrasonic cartridge according to the present invention;

FIG. 3 is a sectional view showing a further embodiment of an ultrasonic cartridge according to the present invention;

FIG. 4 is a schematic view showing a head for ultrasound therapy according to the present invention;

FIGS. 5 and 6 are views showing a state of use of the ultrasonic cartridge and the head for ultrasound therapy using the same according to the present invention.

### DESCRIPTION OF REFERENCE CHARACTERS OF IMPORTANT PARTS

1: head body 10: cartridge housing
11: upper housing member 11a: upper surface portion
11b: first side portion 11c: hollow portion
11d: bellows member 12: lower housing member
12a: lower surface portion 12b: second side portion
13: ultrasonic wave transmission window 14: sealing O-ring member
15: stop groove 16: stopping portion
17: stopper protrusion 18: stopper stopping portion
20: ultrasonic transducer unit 30: pressure regulating valve

### BEST MODE

Hereinbelow, the present invention will be described in detail.

Preferred embodiments of the present invention will be described in detail with reference to the accompanying drawings as follows. Prior to the detailed description of the present invention, it should be noted that the terms and words used in the specification and the claims should not be construed as being limited to ordinary meanings or dictionary definitions. Accordingly, it should be appreciated that there may be various equivalents and modifications that can replace the embodiments and the configurations at the time at which the present application is filed.

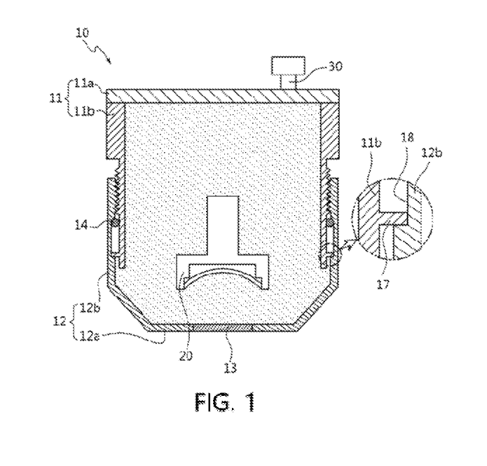

FIG. 1 is a sectional view showing an embodiment of an ultrasonic cartridge according to the present invention, and the embodiment of the ultrasonic cartridge according to the present invention will now be described in detail with reference to FIG. 1.

The embodiment of the ultrasonic cartridge according to the present invention includes a cartridge housing 10 provided at a lower portion thereof with an ultrasonic wave transmission window 13, through which ultrasonic waves are transmitted, and that is configured to be brought in direct contact with skin, with a liquid stored therein.

The cartridge housing 10 is filled with the liquid that serves as a medium of ultrasonic waves and minimizes the loss of ultrasonic waves. In the ultrasonic cartridge according to the present invention, the liquid is water having a low ultrasonic loss coefficient, but may be any liquid that serves as a medium for transmitting the ultrasonic waves other than water.

The cartridge housing 10 is provided at the lower portion thereof with the ultrasonic wave transmission window 13 allowing ultrasonic waves generated from an ultrasonic transducer unit 20 to be transmitted to the skin.

The ultrasonic wave transmission window 13 is, for example, a transparent or semitransparent synthetic resin film, is provided to cover the open lower portion of the cartridge housing 10, serves to transmit ultrasonic waves to a treatment target area, and is configured such that the upper surface thereof is formed to be flat.

The ultrasonic wave transmission window 13 may be modified to be any form capable of transmitting ultrasonic waves to the treatment target area.

The cartridge housing 10 is provided therein with the ultrasonic transducer unit 20 configured to provide ultrasonic waves to the treatment target area through the ultrasonic waves transmission window 13. The ultrasonic transducer unit 20 includes an ultrasonic transducer having a concave hemispherical groove shape to focus the ultrasonic waves on the treatment target area. The ultrasonic transducer is a well-known technology, and a detailed description thereof is omitted.

The cartridge housing 10 includes an upper housing member 11 in which the ultrasonic transducer unit 20 is provided, and a lower housing member 12 in which the ultrasonic wave transmission window 13 is provided.

The lower housing member 12 is moveably coupled to the upper housing member 11 to be capable of adjusting the distance between the ultrasonic transducer unit 20 and the ultrasonic wave transmission window 13.

During the ultrasound therapy, the ultrasonic wave transmission window 13 is brought in contact with skin to be treated, and the lower housing member 12 is coupled to the upper housing member 11 to be moveable in a vertical direction based on a contact surface with the skin, that is, to be moveable up and down.

The up and down direction is a vertical direction based on a reference plane when the contact surface of the lower surface of the cartridge housing 10 brought in contact with the skin to be treated or a skin surface of the treatment target area is set as the reference plane, and does not mean an absolute up and down direction, so it may be variously understood based on the contact surface.

In other words, the cartridge housing 10 moves the lower housing member 12 in the vertical direction based on the lower surface of the ultrasonic wave transmission window 13 brought in contact with the skin, thereby adjusting the distance between the ultrasonic transducer unit 20 and the ultrasonic wave transmission window 13.

To be more specific, by adjusting the distance between an oscillating surface of the ultrasonic transducer where the ultrasonic waves are oscillated and the ultrasonic wave transmission window 13, the depth of focus of the ultrasound formed in the skin is adjusted.

The upper housing member 11 may include: an upper surface portion 11a in which the ultrasonic transducer unit 20 is provided; and a first side portion 11b protruding downward from an outer edge of the upper surface portion 11a. The lower housing member 12 may include: a lower surface portion 12a in which the ultrasonic wave transmission window 13 is provided; and a second side portion 12b protruding upward from an outer edge of the lower surface portion 12a to be overlapped with at least a part of the first side portion 11b.

The upper housing member 11 and the lower housing member 12 are disposed such that the first side portion 11b and the second side portion 12b are overlapped and brought in close contact with each other, whereby the outer circumferential surface or the inner circumferential surface of the first side portion 11b serves as a guide surface that guides the movement of the second side portion 12b.

Further, a stopper protrusion 17 is provided in one of the first side portion 11b and the second side portion 12b to be stopped at an uppermost end and a lowermost end of the lower housing member 12, and a stopper stopping portion 18 is provided in the remaining one of the first side portion lib and the second side portion 12b to be stopped by the stopper protrusion 17 at the uppermost end and the lowermost end of the lower housing member 12, thereby preventing the lower housing member 12 from being excessively moved up or moved down.

As an example, the stopper protrusion 17 may protrude from the outer circumferential surface of the first side portion 11b, and the stopper stopping portion 18 may be formed in a groove shape such that the stopper protrusion 17 is moved by being inserted into the inner circumferential surface of the second side portion 12b, whereby the stopper protrusion 17 is stopped at the upper portion and lower portion of the groove.

The stopper protrusion 17 and the stopper stopping portion 18 may be modified to various other embodiments that limit the movement of the lower housing member 12 by the stopper protrusion 17 being stopped by the stopper stopping portion 18 at the uppermost end and the lowermost end of the lower housing member 12.

The embodiment of the ultrasonic cartridge according to the present invention is configured such that the second side portion 12b is coupled to surround the outer circumferential surface of the first side portion 11b, and although not shown, the first side portion 11b may be coupled to surround the outer circumferential surface of the second side portion 12b.

Further, it is preferable that a sealing O-ring member 14 is inserted between the first side portion 11b and the second side portion 12b.

The sealing O-ring member 14 seals between the first side portion 11b and the second side portion 12b to prevent the liquid stored thereinside, i.e., the medium, from leaking.

It is preferable that the upper housing member 11 is provided with a pressure regulating valve 30 to regulate internal pressure when the lower housing member 12 is moved.

For example, the pressure regulating valve 30 may be provided in the upper surface portion 11a.

The pressure regulating valve 30 is configured to introduce air into the upper housing member 11 when the lower housing member 12 is moved downward, that is, in a direction of increasing the distance between the ultrasonic transducer unit 20 and the ultrasonic wave transmission window 13, and discharge air in the upper housing member 11 when the lower housing member 12 is moved upward, that is, in a direction of decreasing the distance between the ultrasonic transducer unit 20 and the ultrasonic wave transmission window 13.

The liquid is filled in the cartridge housing 10 when the lower housing member 12 is at the highest position, that is, at the highest upward position, and when the lower housing member 12 is moved downward, that is, moved toward the skin and thus the volume of the inside of the cartridge housing 10 is increased, the increased space is filled with air as the air is introduced through the pressure regulating valve.

The pressure regulating valve 30 may be variously modified to any known valve that is opened by a suction force generated when the lower housing member 12 is moved downward such that air is sucked into the cartridge housing 10, and is opened by a pressing force generated when the lower housing member 12 is moved upward such that the air in the cartridge housing 10 is discharged.

Further, the pressure regulating valve 30 may be an electronic valve configured to sense the movement of the lower housing member 12, and be opened only when the lower housing member 12 is moved and be closed when the lower housing member 12 is not moved, that is, stops moving.

The pressure regulating valve 30 may be variously modified to any known valve that is opened when the lower housing member 12 is moved and is closed when the lower housing member stops moving, thereby regulating the internal pressure of the cartridge housing 10.

The lower housing member 12, for example, is threaded with the upper housing member 11 to be moved according to a rotational direction.

To be more specific, the first side portion 11b and the second side portion 12b are threaded with each other, whereby the second side portion 12b is vertically moved along the outer circumferential surface or the inner circumferential surface of the first side portion 11b according to the rotational direction, so as to adjust the distance between the ultrasonic transducer unit 20 and the ultrasonic wave transmission window 13.

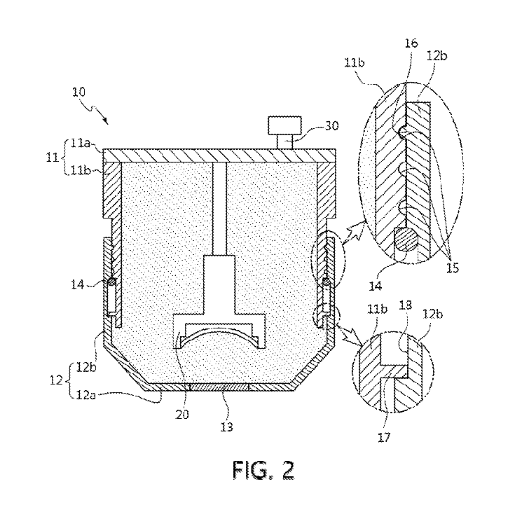

Meanwhile, FIG. 2 is a sectional view showing another embodiment of an ultrasonic cartridge according to the present invention. Referring to FIG. 2, in another embodiment of the ultrasonic cartridge according to the present invention, a stop groove 15 is provided in one of the first side portion 11b and the second side portion 12b, and a stopping portion 16 is provided in the remaining one of the first side portion 11b and the second side portion 12b to be stopped by being engaged with the stop groove 15, wherein at least one of the stop groove 15 and the stopping portion 16 may be provided in plural and spaced apart from each other in a moving direction of the lower housing member 12.

The embodiment of the ultrasonic cartridge according to the present invention shown in FIG. 2 is configured such that multiple stop grooves 15 are provided in the outer circumferential surface of the first side portion 11b while being spaced apart from each other in the moving direction of the lower housing member 12, and stopping portions 16 are provided in the inner circumferential surface of the second side portion 12b.

Although not shown, the stopping portion 16 may be provided in the outer circumferential surface of the first side portion 11b, and the multiple stop grooves 15 may provided in the inner circumferential surface of the first side portion 11b while being spaced apart from each other in the moving direction of the lower housing member 12.

Further, as an example, the multiple stopping portions 16 are provided on the outer circumferential surface of the first side portion 11b while being spaced apart from each other in the moving direction of the lower housing member 12, and the stop groove 15 is provided in the inner circumferential surface of the second side portion 12b.

Further, the stop groove 15 may be provided in the outer circumferential surface of the first side portion 11b, and the multiple stopping portions 16 may be provided on the inner circumferential surface of the first side portion 11b while being spaced apart from each other in the moving direction of the lower housing member 12.

The stop groove 15 and the stopping portion 16 may be modified in various ways so that the position thereof can be locked at a plurality of locations when the lower housing member 12 is moved.

The stopping portion 16 is a hemispherical protrusion, and the stop groove 15 is a hemispherical groove corresponding to the stopping portion 16, wherein the stopping portion 16 may be separated from the stop groove 15 in a state where the stopping portion 16 is inserted into the stop groove 15 when a force exceeding a predetermined level is applied.

In other words, the lower housing member 12 is held in a fixed position as the stopping portion 16 is inserted into the stop groove 15.

Further, when the lower housing member 12 is pushed by applying a force exceeding a preset level, that is, a force more than allowing the stopping portion 16 to be separated from the stop groove 15, the lower housing member 12 is moved as the stopping portion 16 is separated from the stop groove 15, and the stopping portion 16 is inserted in another stop groove 15 or the stop groove 15 is engaged with another stopping portion 16, whereby the moved position of the lower housing member 12 is locked again.

The lower housing member 12 is moved and locked at a position where the stopping portion 16 is inserted into the stop groove 15, and according to a position where the stopping portion 16 is inserted into the stop groove 15, the distance between the ultrasonic transducer unit 20 and the ultrasonic wave transmission window 13 is adjusted.

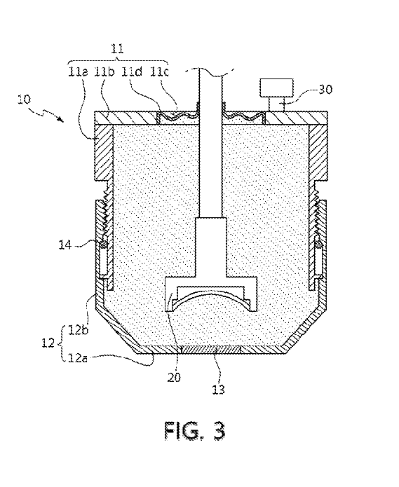

FIG. 3 is a sectional view showing a further embodiment of an ultrasonic cartridge according to the present invention. Referring to FIG. 3, the upper surface portion 11a is provided with a hollow portion 11c, and the hollow portion 11c may be provided with a bellows member 11d configured such that the outer surface thereof is fixed to the upper surface portion 11a and the inner surface thereof surrounds and fixes the outer circumferential surface of the ultrasonic transducer unit 20, whereby a bellows member is freely folded and unfolded and seals the hollow portion 11c.

Since the bellows member 11d is freely folded and unfolded, the ultrasonic transducer unit 20 can be freely moved by the operation of a transducer actuator unit to be described later.

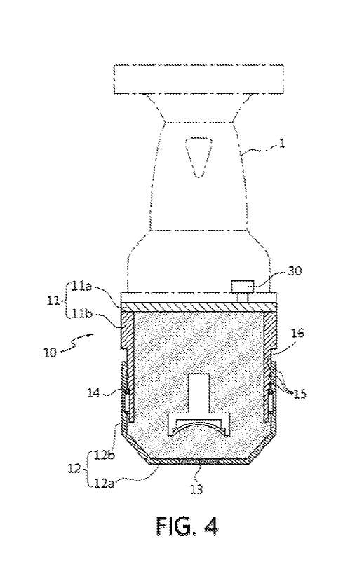

Meanwhile, FIG. 4 is a schematic view showing a head for ultrasound therapy according to the present invention, and the ultrasonic cartridge according to the present invention is detachably mounted to the lower surface of the head body of the head for ultrasound therapy.

The head body of the head for ultrasound therapy is configured in a shape that allows an operator (e.g. a doctor) to grip the head body by hand and bring the same in contact with the patient's skin, to facilitate treatment.

The head body may be provided with a transducer actuator unit (not shown) that can move the ultrasonic transducer unit 20, wherein the transducer actuator unit (not shown) pivots the ultrasonic transducer unit 20 or to moves the ultrasonic transducer unit 20 in the X- and Y-axis directions, that is, in a direction parallel to the contact surface of the skin to be treated, which may be variously modified by a known technique.

The skin, which is the treatment target area, has a flat shape, such as a thigh, and may be a region having a wide skin area.

However, in the case of a region having a curved shape such as abdomen or buttocks, it is preferable that the operator lightly presses the treatment target area of the subject with the lower portion of the head (lower surface of a cartridge 200) to flatten the same.

The head for ultrasound therapy according to the present invention may be configured to irradiate ultrasound waves to any one of the epidermis, dermis, and subcutaneous fat in the skin to position the focus of the ultrasound waves while being moved along the skin to irradiate the ultrasound waves in the state where the lower surface, more specifically, the ultrasonic wave transmission window 13 of the cartridge housing 10 is brought in close contact with the skin.

When the head for ultrasound therapy according to the present invention is implemented as a portable standalone type, the head body may be provided therein with a controller configured to control the operation of the ultrasonic transducer and the operation of the transducer actuator unit for moving the ultrasonic transducer when power is supplied. The head for ultrasound therapy may be operated independently by the control of the controller.

For stable power supply and easy operation, it is preferable to provide a separate controller outside, which controls the operation of the ultrasonic transducer and the operation of the transducer actuator unit for moving the ultrasonic transducer.

As described above, when the controller is separately provided outside, the head body 100 and the external controller may be connected by a cable or the like.

The head body 100 is provided with a touch screen (not shown) for providing an interface with a user, and a control board (not shown) for controlling the operation of the transducer actuator unit and the operation of the ultrasonic transducer by running the software programmed according to a predetermined operating algorithm.

The head body 100 may be provided with a sensor to check whether the ultrasonic cartridge of the present invention, that is, the cartridge housing 10 is properly coupled thereto, such that when the cartridge housing 10 is not properly connected, the signal received from the sensor is output as beep sound or the like to be easily recognized by the operator, i.e., the doctor.

However, it is preferable that the transducer actuator unit (not shown) does not include a structure that moves the ultrasonic transducer unit 20 in the moving direction of the lower housing member 12, that is, in the vertical direction based on a contact surface when the contact surface of the lower surface of the cartridge housing 10 brought in contact with the skin to be treated or a skin surface of the treatment target area is set as the reference plane. This is because the depth of focus of the ultrasound in the skin, which is generated from the ultrasonic transducer unit 20, can be adjusted by the above described ultrasonic cartridge according to the present invention.

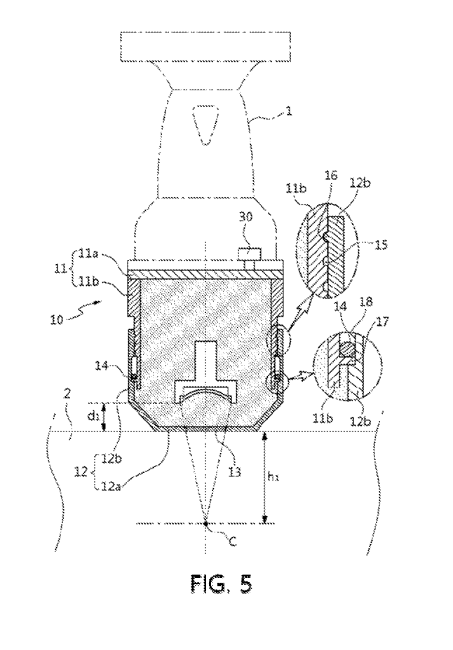

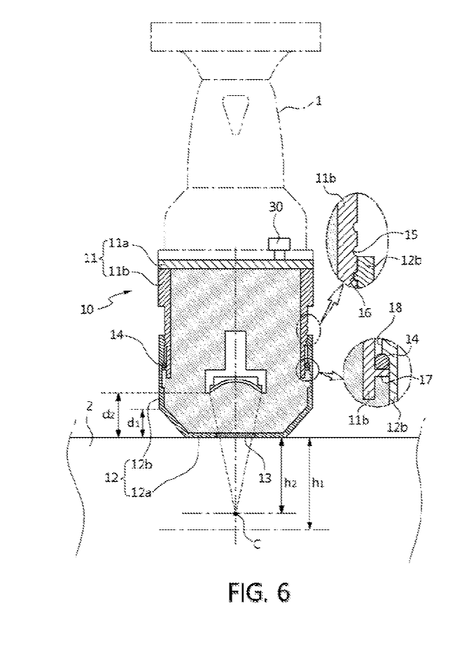

FIGS. 5 and 6 are views showing a state of use of the ultrasonic cartridge and the head for ultrasound therapy using the same according to the present invention. In FIGS. 5 and 6, the head for ultrasound therapy using the ultrasonic cartridge according to the present invention is configured, for example, such that in the state where the lower surface of the lower housing member 12, that is, the lower surface of the ultrasonic wave transmission window 13 is brought in close contact with the corresponding target skin 2, the ultrasonic waves are generated from the ultrasonic transducer unit 20 to treat the skin 2.

FIG. 5 shows an example where the lower housing member 12 is disposed at the uppermost end so the distance d₁ between the ultrasonic transducer unit 20 and the ultrasonic wave transmission window 13 is closest.

Referring to FIG. 5, in the case where the lower housing member 12 is disposed at the uppermost end so the distance d₁ between the ultrasonic transducer unit 20 and the ultrasonic wave transmission window 13 is closest, the focus C of the ultrasound generated from the ultrasonic transducer unit 20 is located at the deepest depth h₁ with respect to the surface of the skin 2.

On the contrary to FIG. 5, FIG. 6 shows an example where the lower housing member 12 is disposed at the lowermost end so the distance d₂ between the ultrasonic transducer unit 20 and the ultrasonic wave transmission window 13 is farthest.

Referring to FIG. 6, in the case where the lower housing member 12 is disposed at the lowermost end so the distance d₂ between the ultrasonic transducer unit 20 and the ultrasonic wave transmission window 13 is farthest, the focus C of the ultrasound generated from the ultrasonic transducer unit 20 is located at the shallowest depth h₂ with respect to the surface of the skin 2.

In other words, referring to FIGS. 5 and 6, the ultrasonic cartridge and the head for ultrasound therapy using the same according to the present invention are configured such that when the distance between the ultrasonic transducer unit 20 and the ultrasonic wave transmission window 13 is decreased by moving the lower housing member 12 upward, the focus C of the ultrasound is moved to the deeper position h₁ in the skin 2, and when the distance between the ultrasonic transducer unit 20 and the ultrasonic wave transmission window 13 is increased by moving the lower housing member 12 downward, the focus C of the ultrasound is moved to the shallower position h₂ in the skin 2.

Thus, the ultrasonic cartridge and the head for ultrasound therapy using the same according to the present invention are configured to adjust the entire length of the cartridge housing 10 such that the distance between the ultrasonic transducer unit 20 and the ultrasonic wave transmission window 13 is adjusted, whereby the depth of focus C of the ultrasound of the skin 2 can be adjusted.

The present invention is configured such that a distance between the contact surface with skin and the ultrasonic transducer is adjusted with a simple housing structure, without an actuator, thereby adjusting the depth of focus of the ultrasound in the skin, and thus, it is possible to significantly simplify the structure of the product and possible to considerably reduce the manufacturing cost.

It is to be understood that the present invention is not limited to the above described embodiments but may be variously modified and embodied within the scope of the present invention without departing from the gist of the present invention.

1. An ultrasonic cartridge comprising:
a cartridge housing provided at a lower portion thereof with an ultrasonic wave transmission window through which ultrasonic waves are transmitted, and configured such that a liquid is stored therein; and
an ultrasonic transducer unit provided in the cartridge housing, and configured to provide ultrasonic waves to a treatment target area through the ultrasonic wave transmission window and form a focus on the treatment target area,
wherein the cartridge housing includes an upper housing member in which the ultrasonic transducer unit is provided, and a lower housing member in which the ultrasonic wave transmission window is provided, and is configured to adjust a distance between the ultrasonic transducer unit and the ultrasonic wave transmission window.

2. The ultrasonic cartridge of claim 1, wherein the lower housing member is moveably coupled to the upper housing member to adjust the distance between the ultrasonic transducer unit and the ultrasonic wave transmission window.

3. The ultrasonic cartridge of claim 2, wherein the lower housing member is moveable in a vertical direction based on a reference plane when a contact surface of a lower surface of the cartridge housing brought in contact with skin to be treated or a skin surface of the treatment target area is set as the reference plane.

4. The ultrasonic cartridge of claim 1, wherein the lower housing member is threaded with the upper housing member to be moved according to a rotational direction.

5. The ultrasonic cartridge of claim 1, wherein the upper housing member includes an upper surface portion in which the ultrasonic transducer unit is provided, and a first side portion protruding downward from an outer edge of the upper surface portion,
the lower housing member includes a lower surface portion in which the ultrasonic wave transmission window is provided, and a second side portion protruding upward from an outer edge of the lower surface portion to be overlapped with at least a part of the first side portion, and
a sealing O-ring member is inserted between the first side portion and the second side portion.

6. The ultrasonic cartridge of claim 5, wherein a stop groove is provided in one of the first side portion and the second side portion,
a stopping portion is provided in a remaining one of the first side portion and the second side portion to be stopped by being engaged with the stop groove, and
at least one of the stop groove and the stopping portion is provided in plural to be spaced apart from each other in a moving direction of the lower housing member.

7. The ultrasonic cartridge of claim 5, wherein a stopper protrusion is provided in one of the first side portion and the second side portion to be stopped at an uppermost end and a lowermost end of the lower housing member, and
a stopper stopping portion is provided in a remaining one of the first side portion and the second side portion to be stopped by the stopper protrusion at the uppermost end and the lowermost end of the lower housing member.

8. The ultrasonic cartridge of claim 2, wherein the upper housing member is provided with a pressure regulating valve to regulate internal pressure when the lower housing member is moved.

9. The ultrasonic cartridge of claim 8, wherein the pressure regulating valve is configured to introduce air into the upper housing member when the lower housing member is moved in a direction of increasing the distance between the ultrasonic transducer unit and the ultrasonic wave transmission window, and discharge air in the upper housing member to an outside when the lower housing member is moved in a direction of decreasing the distance between the ultrasonic transducer unit and the ultrasonic wave transmission window.

10. A head for ultrasound therapy, the head comprising:
an ultrasonic cartridge of claim 1; and
a head body configured such that the ultrasonic cartridge is detachably coupled to a lower portion thereof, and formed in a shape that allows an operator to grip the head body.

11. A head for ultrasound therapy, the head comprising:
an ultrasonic cartridge of claim 2; and
a head body configured such that the ultrasonic cartridge is detachably coupled to a lower portion thereof, and formed in a shape that allows an operator to grip the head body.

12. A head for ultrasound therapy, the head comprising:
an ultrasonic cartridge of claim 4; and
a head body configured such that the ultrasonic cartridge is detachably coupled to a lower portion thereof, and formed in a shape that allows an operator to grip the head body.

13. A head for ultrasound therapy, the head comprising:
an ultrasonic cartridge of claim 5; and
a head body configured such that the ultrasonic cartridge is detachably coupled to a lower portion thereof, and formed in a shape that allows an operator to grip the head body.
